SEQUENCE LISTING (10 sequences)

SEQ ID NO: 1 - DNA, 20 bp, synthetic construct
  source 1..20: mol_type = other DNA
ccaacagtggcataggcttt

SEQ ID NO: 2 - DNA, 20 bp, synthetic construct
  source 1..20: mol_type = other DNA
ctaccagtacgccagcgagt

SEQ ID NO: 3 - DNA, 20 bp, synthetic construct
  source 1..20: mol_type = other DNA
ggaaaagcggccagtatagg

SEQ ID NO: 4 - DNA, 19 bp, synthetic construct
  source 1..19: mol_type = other DNA
agccctacgagcacctgac

SEQ ID NO: 5 - DNA, 20 bp, synthetic construct
  source 1..20: mol_type = other DNA
atacaagacagctcccaggc

SEQ ID NO: 6 - DNA, 20 bp, synthetic construct
  source 1..20: mol_type = other DNA
tcggactctgcaaggagaac

SEQ ID NO: 7 - DNA, 20 bp, synthetic construct
  source 1..20: mol_type = other DNA
tctggaatacccatccaagg

SEQ ID NO: 8 - DNA, 19 bp, synthetic construct
  source 1..19: mol_type = other DNA
atgcgtcacgctatgtgct

SEQ ID NO: 9 - DNA, 25 bp, synthetic construct
  source 1..25: mol_type = other DNA
tcacccacactgtgcccatctacga

SEQ ID NO: 10 - DNA, 25 bp, synthetic construct
  source 1..25: mol_type = other DNA
cagcggaaccgctcattgccaatgg

(19) United States
(12) Patent Application Publication
(10) Pub. No.: US 2026/0158089 A1
(43) Pub. Date: Jun. 11, 2026

(54) COMPOSITION COMPRISING MANGO EXTRACT FOR PREVENTION OF HAIR LOSS

(71) Applicant: The Industry & Academic Cooperation in Chungnam National University (IAC), Daejeon (KR)

(72) Inventor: Keekwang KIM, Chungcheongnam-do (KR)

(73) Assignee: The Industry & Academic Cooperation in Chungnam National University (IAC), Daejeon (KR)

(21) Appl. No.: 18/686,208

(22) PCT Filed: Jul. 1, 2022
(86) PCT No.: PCT/KR2022/009546

§ 371 Date: Feb. 23, 2024

(30) Foreign Application Priority Data
Aug. 25, 2021 (KR) 10-2021-0112225

(51) Int. Cl. A61K 36/22; A61K 9/00; A61P 17/14

(52) U.S. Cl. CPC A61K 36/22; A61K 9/0056; A61P 17/14

(57) ABSTRACT

The present disclosure relates to a composition including a mango extract and more preferably a solvent extract of mango leaves as an active ingredient for alleviation, suppression, prevention, or treatment of hair loss, and a preparation method therefor. The mango extract according to the present disclosure can be used in a pharmaceutical composition, a food composition, or a cosmetic composition useful for alleviation, suppression, prevention, and treatment of hair loss.

### TECHNICAL FIELD

The present disclosure relates to a composition including a mango extract and more preferably a solvent extract of mango leaves as an active ingredient for alleviation, suppression, prevention, or treatment of hair loss, and a preparation method therefor.

This present application claims benefit of priority to Korean Patent Application No. 10-2021-0112225 filed on Aug. 25, 2021, in the Korean Intellectual Property Office, the entire disclosure of which is incorporated herein by reference.

### BACKGROUND ART

Hair loss is the physiological loss of hair from the hair follicle. Normal hair goes through a cycle of hair growth and hair loss by repeating a 3-year growth period, a 3-week catagen, and a 3-month telogen. Hair loss is broadly divided into two types: natural hair loss, in which telogen hair naturally falls out of the hair follicle, and abnormal hair loss caused by pregnancy, genetic factors, abnormal hormonal secretion, aging, extreme dieting, mental stress, irregular lifestyle, fever, excessive use of chemical products, etc.

As modern people's hair loss is caused by acquired factors, including environmental issues, rather than congenital factors, the domestic hair loss population in 2019, including the potential hair loss population, exceeds 10 million. In addition, research shows that the domestic market, including cosmetics, quasi-drugs, and pharmaceuticals, is worth approximately 4 trillion won. In the past, hair loss was thought to be an issue only for middle-aged people, but currently, it is increasing significantly among young people in their 20s and 30s and is being dismissed as a disease. Due to this increase, research on hair loss prevention, treatment, and hair growth is also increasing.

Dermal papilla cells are known to be cells that secrete several signaling substances that regulate the growth cycle in hair follicles. In addition, testosterone, a steroid sex hormone of the androgen group in the body, acts with 5α-reductase in dermal papilla cells to produce dihydrotestosterone (DHT), which has a direct influence on male pattern baldness hair loss, causing hair to fall out from hair follicles.

Finasteride and dutasteride, which are approved by the FDA and hold the majority of the market share in the hair loss market, induce hair growth and slow the progression of hair loss by inhibiting the activity of 5alpha-reductase type 2. However, these treatments are also chemical drugs and need to be used at the risk of side effects such as allergic contact dermatitis, decreased libido, gynecomastia in men, decreased ejaculate volume, and fatal risks in women of childbearing age.

For this reason, the development of natural product-derived treatments with fewer side effects is essential and requires continued research.

### DISCLOSURE OF INVENTION

### Technical Problem

The present inventors have made extensive research efforts to develop a natural product-derived hair loss treatment with fewer side effects. As a result, the present disclosure was completed by identifying that mango (Mangifera indica) suppresses hair loss-related gene expression and ultimately exhibits a significant hair loss suppression effect.

Accordingly, an aspect of the present disclosure is directed to providing a pharmaceutical composition including a mango (Mangifera indica) extract as an active ingredient for alleviation, prevention, or treatment of hair loss.

Another aspect of the present disclosure directed to providing a food composition including a mango extract as an active ingredient for alleviation or suppression of hair loss.

Yet another aspect of the present disclosure is directed to providing a cosmetic composition including a mango extract as an active ingredient for alleviation or suppression of hair loss.

Yet another aspect of the present disclosure is directed to providing a method for alleviation, prevention, or treatment of hair loss by administering a mango extract in an amount effective for alleviation, prevention, or treatment of hair loss to a subject in need thereof.

Yet another aspect of the present disclosure is directed to a use of a mango extract in alleviation, prevention, or treatment of hair loss.

### Solution to Problem

The present inventors have made extensive research efforts to develop a natural product-derived hair loss treatment with fewer side effects. As a result, it was identified that mango (Mangifera indica) suppressed hair loss-related gene expression and ultimately exhibits a significant hair loss suppression effect.

An embodiment of the present disclosure relates to a composition including a mango extract and more preferably a solvent extract of mango leaves as an active ingredient for alleviation, suppression, prevention, or treatment of hair loss, and a preparation method therefor.

An embodiment of the present disclosure identified significant results in hair loss suppression effect through a genetic analysis experiment related to the hair production cycle in dermal papilla cells using water extracts of Mangifera indica leaves (WEML).

Hereinafter, the present disclosure will be described in more detail.

According to an aspect of the present disclosure, an embodiment of the present disclosure relates to a pharmaceutical composition including a mango (Mangifera indica) extract as an active ingredient for alleviation, prevention, or treatment of hair loss.

In an embodiment of the present disclosure, “mango (Mangifera indica)” is an evergreen dicotyledonous plant of the genus Mangifera of family Anacardiaceae of order Sapindales, and has long spear-shaped leaves of approximately 30 cm in length.

Representative mango leaf components include mangiferin, phenolic constituents, glucose, flavonoids, arabinose, xylose, rhamnose, galactose, leucine, tyrosine, valine, protocatechuic acid, catechin (VI), alanine, glycine, tannins, kinic acid, α-pinene, β-pinene) (VI), etc.

By virtue of the anthocyanins contained in mango leaves, continuous consumption of tea made by heating the leaves has been reported to have positive effects on diabetic retinopathy and diabetic angiopathy, and has been known to have a powerful antioxidant effect, eliminating active oxygen that cause damage to blood vessels and organs.

Apple mango is one of the mango varieties with a similar shape to an apple, and there has been no research on the hair loss prevention function of apple mango leaves to date.

As used herein, the term “extract” has a meaning that is commonly used as a crude extract in the art as described above, and broadly, encompasses a fraction obtained by additionally fractionating the extract. In other words, the mango extract includes not only extracts obtained by using the above-described extraction solvents but also extracts obtained by additionally applying a purification procedure to the same. For example, the mango extract of an embodiment of the present disclosure includes fractions obtained through various purification methods that are additionally performed, such as a fraction obtained by passing the extracts through an ultrafiltration membrane with a predetermined molecular weight cut-off value and a fraction obtained by various types of chromatography (manufactured for separation depending on size, charge, hydrophobicity, or hydrophilicity).

The mango extract used in an embodiment of the present disclosure may be prepared as a powder type extract by additional procedures, such as distillation under reduced pressure and freeze-drying or spray drying.

In an embodiment of the present disclosure, mango roots, stems, and leaves may all be used. For example, mango leaves, preferably apple mango leaves, may be used.

In an embodiment of the present disclosure, an extraction solvent may be one or more solvents selected from the group consisting of water and a straight-chain or branched alcohol having 1 to 4 carbon atoms, but is not limited thereto.

In an embodiment of the present disclosure, the extraction temperature may be, for example, 0° C. to 150° C., specifically 50° C. to 100° C., but is not limited thereto.

In an embodiment of the present disclosure, the extraction time may be, for example, 1 hour to 10 hours, specifically 3 hours to 8 hours, and more specifically 5 hours to 7 hours, but is not limited thereto.

The mango extract of an embodiment of the present disclosure may be extracted by a known natural substance extraction method. For example, the extraction may be carried out by cold extraction, hot-water extraction, ultrasonic extraction, reflux cooling extraction, or heating extraction, and specifically, hot-water extraction and reflux cooling extraction. The extraction may be repeated one to ten times, and more specifically two to seven times.

In an embodiment of the present disclosure, the mango extract may be contained in an amount of 0.001 to 3.0% by weight relative to the total weight of the composition of an embodiment of the present disclosure, and more specifically, 0.01 to 3.0% by weight, 0.1 to 3.0% by weight, and 1.0 to 3.0% by weight relative to the total weight of the composition, but is not limited thereto. When the extract is contained in less than 0.001% of the total weight of the composition, it is difficult to expect an effect, and when the extract is contained in excess of 3.0% by weight, there is an issue with economic feasibility.

Another aspect of the present disclosure, an embodiment of the present disclosure relates to a method for preparing a mango extract having activity for alleviation, suppression, prevention, or treatment of hair loss.

In one embodiment of the present disclosure, the mango extract may be a crude extract obtained by extracting one or more selected from the group consisting of mango leaves, stems, or roots with one or more solvents selected from the group consisting of water or a straight-chain or branched alcohol having 1 to 4 carbon atoms.

The preparation process of the mango extract according to an embodiment of the present disclosure is described in more detail as follows:

Mango leaves are cut into pieces, washed with water to remove foreign matter, and then dried, followed by reflux extraction with an approximate 10 to 30-fold volume, and preferably a 17 to 23-fold volume, of an extraction solvent with respect to the weight of the mango leaves. After extraction, the resulting extract is filtered, thereby collecting a filtrate. An extraction temperature may be 50 to 150° C., and preferably, 80 to 100° C., but an embodiment of the present disclosure is not limited thereto.

An extraction process may be performed once or repeated several times, and according to one exemplary embodiment of the present disclosure, after first extraction, re-extraction may be selected, and this is to prevent a decrease in extraction efficiency only with the first extraction due to loss in the case of a herbal medicine extract being produced in large quantities because of a high moisture content of a herbal medicine although effectively filtered. In addition, as a result of verifying the extraction efficiency for each stage, it was revealed that approximately 80 to 90% of the total extraction amount was obtained through second extraction.

In one embodiment of the present disclosure, when the extraction process is repeated twice, the obtained residue is subjected to reflux extraction again with an approximate 5 to 15-fold volume, and preferably, an 8 to 12-fold volume, of an extraction solvent. After extraction, the resulting product is filtered, combined with the previously-obtained filtrate and concentrated under reduced pressure, thereby preparing a mango extract. As such, extraction efficiency may increase through two-stage extraction and by mixing the filtrates obtained by respective extractions, but the extract of an embodiment of the present disclosure is not limited by the number of extractions.

When the amount of the solvent used in the preparation of the mango extract is too small, stirring is difficult, and extraction efficiency decreases due to low solubility of the extract. When the amount of the solvent used in the preparation of the mango extract is too large, as the amount of a solvent used in a subsequent purification stage becomes larger, the solvent is not economical and may have a handling issue. Accordingly, it is preferable that the amount of a solvent used herein be within the above range.

The content of the mango extract as an active ingredient in the composition of an embodiment of the present disclosure may be appropriately controlled depending on the form and purpose of use, condition of patients, kind and severance of symptoms, etc., and it may be 0.001 to 99.9% by weight or 0.1 to 99.9% by weight, preferably 0.1 to 50% by weight or 0.1 to 40% by weight, based on solid content weight, without being limited thereto.

The composition of an embodiment of the present disclosure may be administered to mammals including humans in various routes. The administration method may be all the commonly used methods, and for example, the composition may be administered through oral, dermal, intravenous, intramuscular, subcutaneous routes, and the like, and preferably, the composition may be orally administered.

The composition of an embodiment of the present disclosure may be used by formulating into oral formulations such as powder, granules, tablets, capsules, ointment, suspension, emulsion, syrup, aerosol, and the like, or parenteral formulations such as percutaneous agents, suppositories, and sterile injection solution, or the like, according to common methods, respectively.

In addition to the extract, the composition of an embodiment of the present disclosure may further include pharmaceutically appropriate and physiologically acceptable adjuvant such as carrier, diluents or excipient.

The carrier, excipient and diluents that may be included in the composition of an embodiment of the present disclosure may include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methylhydroxybenzoate, propylhydroxybenzoate, talc, magnesium stearate, and mineral oil.

In addition, formulations may be prepared by using generally used diluents or excipient such as a filler, a bulking agent, a binder, a wetting agent, a disintegrating agent, surfactant, etc. A solid formulation for oral administration may include a tablet, a pill, powder, granule, a capsule, etc. These solid formulations may be prepared by mixing the extract with one or more excipients such as starch, calcium carbonate, sucrose or lactose, gelatin, etc. In addition to the simple excipients, a lubricant such as magnesium stearate, and talc may also be used.

Formulations for oral administrations include suspensions, oral solutions, emulsions, syrups, and ointments. In addition to generally used simple diluents such as water and liquid paraffin, the liquid formulations may include various excipients such as wetting agents, sweeteners, fragrances and preservatives.

Formulations for parenteral administration may include sterilized aqueous solutions, non-aqueous solvents, suspensions, emulsions, lyophilizing agents, suppositories, epidermal formulations, etc. The non-aqueous solvents and suspensions may include vegetable oils such as propylene glycol, polyethyleneglycol, or olive oil, and injectable esters such as ethyl oleate.

As a base for suppositories, witepsol, macrogol, tween 61, cacao butter, laurinum, glycerogelatin may be used.

In a specific embodiment of applying the composition of an embodiment of the present disclosure to humans, although the extract composition of an embodiment of the present disclosure may be administered alone, the composition may be administered with a generally pharmaceutical carrier selected considering the administration method and standard pharmaceutical practices.

For example, the composition containing the extract of an embodiment of the present disclosure may be administered orally, in an oral cavity or sublingually in a tablet form containing starch or lactose, or in a capsule form with or without excipient, or in an elixir or suspension form containing sweetening or coloring chemical drugs. The liquid base may be formulated with pharmaceutically acceptable additives such as suspension (for example, semisynthesized glyceride such as methylcellulose, witepsol, or a glyceride mixture such as a mixture of apricot kernel oil and PEG-6 ester or a mixture of PEG8 and caprylic/capric glyceride).

A dose of the composition containing the mango extract of an embodiment of the present disclosure may vary depending on the age, body weight, gender of a patient, administration form, health condition and severance of disease. The dose may be administered once or several times a day in divided doses at regular time intervals according to the decision of a doctor or pharmacist. For example, a daily dose may be 0.5 to 500 mg/kg, preferably 0.5 to 300 mg/kg, based on the content of an active ingredient. The above doses are exemplified as an average case, and its dose may increase or decrease depending on personal differences.

When a daily dose of the composition containing the mango extract of an embodiment of the present disclosure is less than the above range, a significant effect may not be obtained. When the daily dose thereof exceeds the above range, not only is it uneconomical, but it is outside the range of the usual dose, so there may be a risk of undesirable side effects, so it is better to keep the daily dose within the above range.

According to yet another aspect of the present disclosure, an embodiment of the present disclosure relates to a food composition including the mango extract for alleviating or suppressing hair loss.

In an embodiment of the present disclosure, food may be various beverages, food additives, etc., but is not limited thereto.

The content of the mango extract as an active ingredient contained in the food composition of an embodiment of the present disclosure is not particularly limited and may appropriately vary depending on the form of food, desired use or the like, and, for example, may be added in an amount of 0.01 to 15% by weight of the total weight of the food, and the health beverage composition may be added in an amount of 0.02 to 10 g, preferably 0.3 to 1 g, based on 100 ml.

In addition to containing the extract as an essential ingredient in the indicated ratio, the food composition of an embodiment of the present disclosure has no particular limitations on the liquid ingredient and may contain various flavoring agents or natural carbohydrates as additional ingredients like conventional beverages.

Examples of the aforementioned natural carbohydrates may be common saccharides such as monosaccharides, for example, glucose, fructose, and the like, disaccharides, for example, maltose, sucrose, and the like, and polysaccharides, for example, dextrin, cyclodextrin, and the like, and sugar alcohols, such as xylitol, sorbitol, erythritol, and the like. As flavoring agents other than those mentioned above, natural flavoring substances (thaumatin, stevia extract (for example, rebaudioside A, glycyrrhizin, etc.) and synthetic flavoring agents (saccharin, aspartame, etc.) may be favorably used. The ratio of the natural carbohydrate is generally about 1 to 20 g, preferably about 5 to 12 g per 100 ml of the composition of an embodiment of the present disclosure.

In addition to the above, the composition of an embodiment of the present disclosure may include various nutrients, vitamins, minerals (electrolyte), flavoring agents such as synthetic flavoring agents and natural flavoring agents, coloring agents and enhancers (cheese, chocolate, etc.), pectic acid and salts thereof, alginic acid and salts thereof, organic acids, protective colloidal thickening agents, pH controlling agents, stabilizing agents, preservatives, glycerin, alcohol, carbonizing agents as used in carbonated beverages, and the like. In addition, the composition of an embodiment of the present disclosure may include fruits, as used in preparing natural fruit juices and fruit juice beverages and vegetable beverages. These components may be used independently or in combination. Although the proportion of these additives is not of great importance, it is generally selected from a range of 0 to about 20 parts by weight per 100 parts by weight of the composition of an embodiment of the present disclosure.

According to yet another aspect of the present disclosure, an embodiment of the present disclosure relates to a cosmetic composition for alleviation or prevention of hair loss including a mango extract as an active ingredient.

The cosmetic composition of an embodiment of the present disclosure may include components conventionally used in a cosmetic composition, in addition to the mango extract, the active ingredient, and such components may include, for example, conventional additives including an antioxidant, a stabilizer, a solubilizer, a vitamin, a pigment, and a fragrance, and a carrier.

The cosmetic composition of an embodiment of the present disclosure may be prepared as any formulation commonly prepared in the art. The cosmetic composition may be formulated as, for example, a solution, a suspension, an emulsion, a paste, a gel, a cream, a lotion, a powder, a soap, a surfactant-containing cleanser, an oil, a pack, a message cream, a spray or the like, but is not limited thereto.

When the formulation of an embodiment of the present disclosure is a paste, a cream or a gel, animal oil, vegetable oil, wax, paraffin, starch, traganth, cellulose derivatives, polyethylene glycol, silicone, bentonite, silica, talc, zinc oxide or the like may be used as the carrier ingredient.

When the formulation of an embodiment of the present disclosure is a solution or an emulsion, a solvent, a solubilizing agent or an emulsifier may be used as the carrier ingredient, and examples thereof include water, ethanol, isopropanol, ethyl carbonate, ethyl acetate, benzyl alcohol, benzyl benzoate, propylene glycol, 1,3-butylglycol oil, glycerol aliphatic esters, polyethylene glycol or sorbitan fatty acid esters.

When the formulation of an embodiment of the present disclosure is a suspension, a liquid diluent such as water, ethanol and propylene glycol, a suspending agent such as ethoxylated isostearyl alcohol, polyoxyethylene sorbitol ester and polypoxyethylene sorbitan ester, microcrystalline cellulose, aluminum metahydroxide, bentonite, agar, traganth or the like may be used as the carrier ingredient.

When the formulation of an embodiment of the present disclosure is a powder or a spray, lactose, talc, silica, aluminum hydroxide, calcium silicate, or polyamide powders may be used as the carrier ingredient, and in particular, when the formulation is a spray, a propellent such as chlorofluorohydrocarbon, propane/butane or dimethyl ether may be contained.

When the formulation of an embodiment of the present disclosure is a surfactant-containing cleanser, aliphatic alcohol sulfate, aliphatic alcohol ether sulfate, sulphosuccinic acid monoester, isethionate, imidazolinium derivative, methyltaurate, sarcosinate, fatty acid amide ether sulfate, alkylamidobetain, aliphatic alcohol, fatty acid glyceride, fatty acid diethanolamide, vegetable oils, a lanolin derivative or ethoxylated glycerol fatty acid ester or the like may be used as the carrier ingredient.

When the cosmetic composition of an embodiment of the present disclosure is a soap, a surfactant-containing cleansing formulation, or a surfactant-free cleansing formulation, the cosmetic composition may be applied to the skin and then wiped off, removed, or washed with water. Specifically, the soap may include liquid soap, soap powder, solid soap, and oil soap; the surfactant-containing cleansing formulation may include cleansing foam, cleansing water, cleansing towel, and cleansing pack; and the surfactant-free cleansing formulation may include cleansing cream, cleansing lotion, cleansing water, and cleansing gel, without being limited thereto.

When each composition of an embodiment of the present disclosure is administered/applied to the human body, it is deemed that there is no concern about side effects compared to other synthetic products in light of the general characteristics of natural extracts. In reality, as a result of toxicity tests on standardized compositions, there was no influence on the living body.

According to yet another aspect of the present disclosure, an embodiment of the present disclosure relates to a method for treating hair loss, wherein the method includes administering a mango extract in an amount effective for alleviation, prevention or treatment of hair loss to a subject in need thereof.

As used herein, the term “subject” refers to a target in need of treatment of a disease (hair loss), and more specifically, a human, or a mammal such as a non-human primate, a mouse, a rat, a dog, a cat, a horse or a cow.

According to yet another aspect of the present disclosure, an embodiment of the present disclosure relates to an use of a mango extract in alleviation, prevention, or treatment of hair loss.

The mango extract is the same as the active ingredient of the pharmaceutical composition for alleviation, prevention or treatment of hair loss of an embodiment of the present disclosure described above, so that a description of overlapping contents therebetween is omitted to avoid excessive complexity of the present specification.

### Advantageous Effects of Invention

An embodiment of the present disclosure relates to a composition including a mango extract and more preferably a solvent extract of mango leaves as an active ingredient for alleviation, suppression, prevention, or treatment of hair loss, and a preparation method therefor. The mango extract according to an embodiment of the present disclosure can be used in a pharmaceutical composition, a food composition, or a cosmetic composition useful for alleviation, suppression, prevention, and treatment of hair loss.

### BRIEF DESCRIPTION OF DRAWINGS

FIG. 1 illustrates the results of evaluating the active oxygen scavenging ability of a hot water extract of mango leaves according to an embodiment of the present disclosure.

FIG. 2 illustrates the cell viability evaluation results of the hot water extract of mango leaves according to an embodiment of the present disclosure.

FIGS. 3A to 3E illustrate the influence of the hot water extract of mango leaves of an embodiment of the present disclosure on hair loss-related gene expression according to an embodiment of the present disclosure. (FIG. 3A: EGR1, FIG. 3B: SGK, FIG. 3C: DKK1, FIG. 3D: SRD5A1, and FIG. 3E: SRD5A2)

### DESCRIPTION OF EMBODIMENTS

Hereinafter, the present disclosure will be described in more detail through examples. These examples are only for illustrating the present disclosure in more detail, and it will be apparent to those skilled in the art that the scope of the present disclosure is not to be construed as being limited by these examples according to the gist of the present disclosure.

The results of all experiments were expressed as Mean±Standard error, and the statistical analysis of the experimental data was performed using Student's t-test to determine the significance of the experimental group relative to the control group. It was determined to be statistically significant when the p value was less than 0.001.

### Preparation Example. Preparation of Mango Hot Water Extract of an Embodiment of the Present Disclosure

Apple mango leaves (Mangifera indica leaves) used in the experiment were obtained from a mango orchard in Geumsan-gun, Chungcheongnam-do in January 2021.

100 g of apple mango leaves were placed in a reflux extractor along with 2 L of primary distilled water. After heating the decoction to 100° C. for 6 hours from the point of boiling, a reduced-pressure filtrate was produced on filter paper (Advantec No. 2, Japan). Next, the concentrate was obtained using a rotary vacuum evaporator (Eyela, Japan), and dried for 7 days using a freeze dryer (IlshineBioBase, Korea) to produce a dried powder. The final freeze-dried extract was 11.254 g, and the yield was identified to be 11.254%.

### Experimental Example 1. Evaluation of Antioxidant Effect (Active Oxygen Scavenging Ability)—ABTS Assay

First, 7 mM of ABTS (2,2′-azino-bis-3-ethylbenzothi'azoline-6-sulphonic acid) solution and 2.4 mM of potassium persulfate solution were mixed in equal volumes (1:1), and the reaction was induced at room temperature under light blocking conditions for 24 hours to prepare an ABTS solution in a free radical state.

Next, 80 μl of the prepared ABTS solution in a free radical state was added to a 96-well plate, and diluted with sterilized distilled water. When the absorbance was measured at a wavelength of 734 nm using a microplate reader (Molecular Devices EMax Plus, USA), an ABTS working solution was prepared so that the measured value was around 0.7.

Finally, 20 μl of mango leaf hot water extract (WEML) (25, 50 or 100 μg/ml) of an embodiment of the present disclosure and 80 μl of ABTS working solution were added to a 96-well plate and reacted at room temperature for 5 minutes, and then the absorbance was measured at a wavelength of 734 nm. In this connection, resveratrol (25 or 50 μM), an antioxidant known to have excellent active oxygen scavenging ability, was set as a positive control group. Antioxidant efficacy (ABTS radical scavenging activity (%)) was calculated according to Equation 1 below.

$$ABTS radical scavering activity ( \% ) = ( 1 - \frac{{A}_{Sample} - {A}_{Sampleblank}}{{A}_{Blank}} ) \times 100 \qquad [ Equation 1 ]$$

A_{Sample}: Absorbance of sample and ABTS reaction
A_{Sampleblank}: Absorbance of sample and DW reaction
A_{Blank}: Absorbance of ABTS and DW reaction

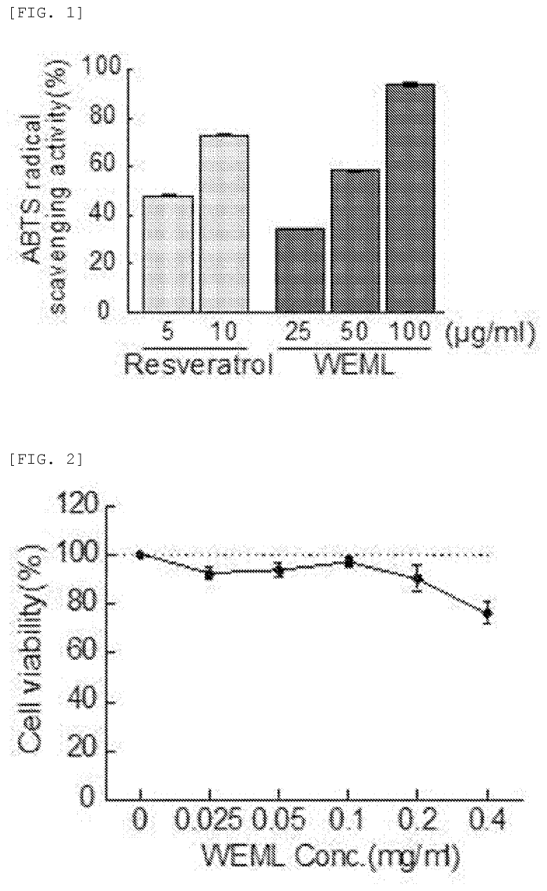

As can be identified in FIG. 1, the ABTS radical scavenging ability of the mango leaf hot water extract of an embodiment of the present disclosure at a concentration of 100 μg/ml was 93.4%, exhibiting a superior active oxygen scavenging ability as compared to the positive control group, resveratrol (47.8% at a concentration of 5 μg/ml, 72.9% at a concentration of 10 μg/ml).

### Experimental Example 2. Measurement of Cell Viability—MTS Assay

Dermal papilla cells (DP) received from the Department of Dermatology at Chungnam National University College of Medicine were dispensed in a 96-well plate at 1×10⁴ cells per well. The cells were cultured in a DMEM/F12 medium (Welgene, Korea) with a culture solution supplemented with 10% FBS (Welgene, Korea), 1% 100 U/ml penicillin, and 100 μg/ml streptomycin (Welgene, Korea) for 24 hours in a CO₂ incubator (Thermo, USA) under the standard cell culture conditions of 5% CO₂ and 37° C. Next, each well was treated with the mango leaf hot water extract (WEML) of an embodiment of the present disclosure at different concentrations (25, 50, 100, 200 or 400 μg/ml) and cultured in the same manner for 36 hours. Finally, 20 μl of MTS (3-(4,5-dimethylthiazol-2-yl)-5-(3-carboxymethoxyphenyl)-2-(4-sulfophenyl)-2H-tetrazolium) reagent (Promega, USA) was added. Absorbance was measured at a wavelength of 490 nm. Cell viability (%) was calculated according to Equation 2 below.

$$Cell viability ( \% ) = \{ ( absorbance of sample added group - absorbance of the sample itself ) / absorbance of control group \} \times 100 \qquad [ Equation 2 ]$$

As can be identified in FIG. 2, at concentrations of 0.025, 0.05, 0.1, and 0.2 mg/ml of mango leaf hot water extract of an embodiment of the present disclosure, an average cell viability of 90.0% was observed, whereas at a concentration of 0.4 mg/ml, it was identified to decrease to 77.5%. Accordingly, subsequent experiments were performed within the concentration range (˜0.2 mg/ml) that did not cause cytotoxicity.

### Experimental Example 3. Gene Expression Analysis

Dermal papilla cells were dispensed in a 6-well plate at 2×10⁵ cells per well. The cells were cultured in a DMEM/F12 medium with a culture solution supplemented with 10% FBS, 1% 100 U/ml penicillin, and 100 μg/ml streptomycin for 24 hours in a CO₂ incubator under the conditions of 5% CO₂ and 37° C. Next, the mango leaf hot water extract of an embodiment of the present disclosure was treated at a concentration of 0.1 mg/ml and further cultured for 36 hours. In this connection, a sample that was not treated with the mango leaf hot water extract of an embodiment of the present disclosure was set as a control group.

The culture solution was removed, the dermal papilla cells were washed with DPBS, and RNA was extracted using the eCube Tissue RNA Mini Kit (PhileKorea, Korea). The extracted RNA was quantified using a Qubit 2.0 Fluorometer (Invitrogen, USA), and 1 μl of random hexamer (100 pmol/μl) and 1 μl of dNTP mix (10 mM) were added to 1 μg of RNA, and the total volume was adjusted to 8 μl with DEPC-treated water. Next, the reaction was performed in a constant temperature water bath at 65° C. for 5 minutes and then immediately cooled in ice.

To each plate, 4 μl of M-MLV RT reaction buffer (Promega, USA), 1 μl of RNase inhibitor (Enzynomics, Korea), 1 μl of M-MLV reverse transcriptase (Promega, USA), and 4 μl of DEPC-treated water were added to make a total volume of 10 μl.

CDNA synthesis was performed at room temperature for 10 minutes and then at 50° C. for 1 hour and diluted to 1/5 using D.W. 5 μl of cDNA, 2 μl of D.W., 10 μl of 2× Prime Q-mater Mix (Geneybio, Korea), and 1.5 μl each of the forward/reverse primers (10 pmol/μl) in Table 1 below were mixed and then used.

TABLE 1

| | Forward/reverse | |
|---|---|---|
| Gene | (SEQ ID NO. ) | Primer sequence (5′ to 3′) |
| SRD5A1 | forward (SEQ ID NO. 1) | CCA ACA GTG GCA TAG GCT TT |
| | reverse (SEQ ID NO. 2) | CTA CCA GTA CGC CAG CGA GT |
| EGR1 | forward (SEQ ID NO. 3) | GGA AAA GCG GCC AGT ATA GG |
| | reverse (SEQ ID NO. 4) | AGC CCT ACG AGC ACC TGA C |
| SGK | forward (SEQ ID NO. 5) | ATA CAA GAC AGC TCC CAG GC |
| | reverse (SEQ ID NO. 6) | |
| DKK1 | forward (SEQ ID NO. 7) | TCT GGA ATA CCC ATC CAA GG |
| | reverse (SEQ ID NO. 8) | ATG CGT CAC GCT ATG TGC T |
| β-actin | forward (SEQ ID NO. 9) | TCA CCC ACA CTG TGC CCA TCT |
| | | ACG A |
| | reverse (SEQ ID NO. 10) | CAG CGG AAC CGC TCA TTG CCA |
| | | ATG G |

qRT-PCR identified changes in mRNA expression of BMP6, CTNNB1, SRD5A1, SRD5A2, EGR1, SGK, and DKK1 using AriaMx. The resulting value was corrected using the mRNA expression level of β-actin.

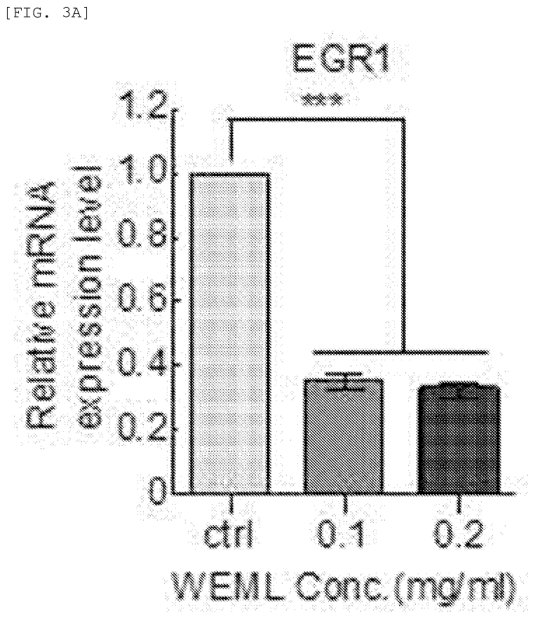

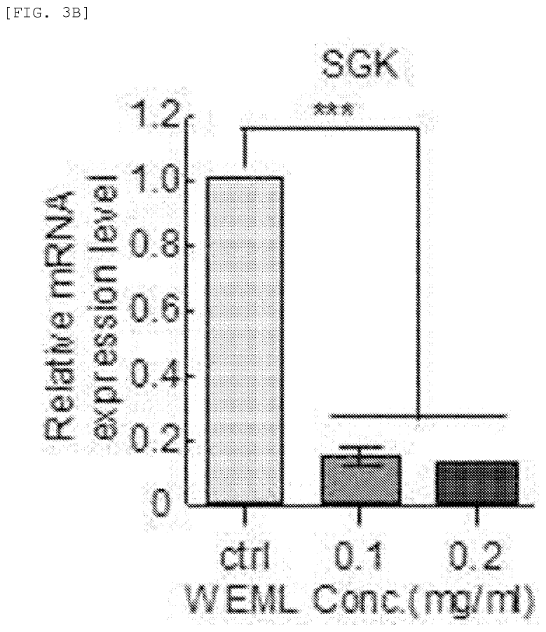

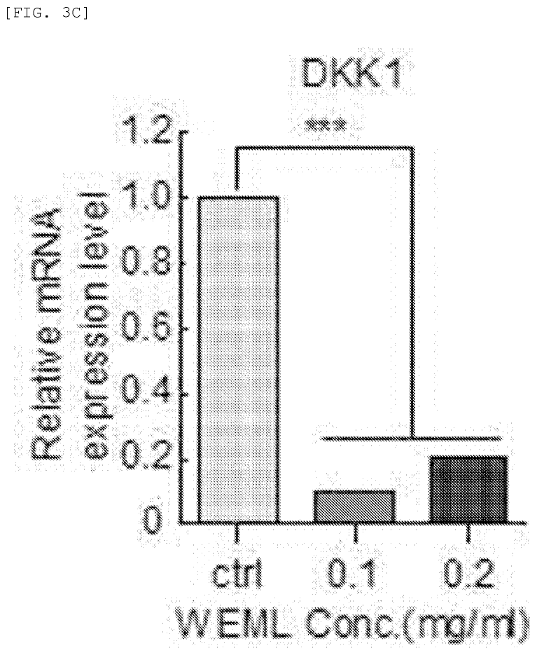

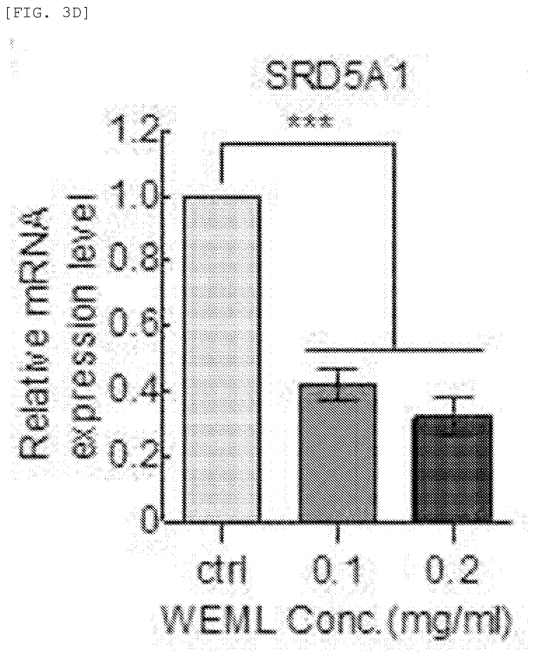

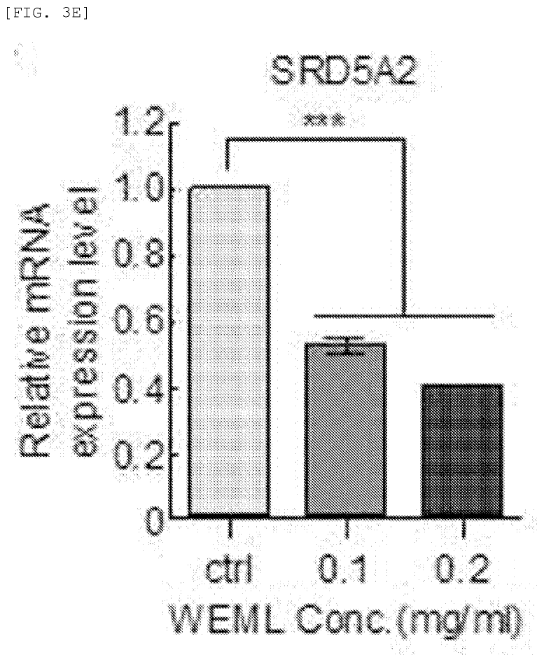

As can be identified in FIGS. 3A to 3E, when treated with the mango leaf hot water extract of an embodiment of the present disclosure at a concentration of 0.1 mg/ml, the mRNA expression levels of EGR1 (FIG. 3A), SGK (FIG. 3B), and DKK1 (FIG. 3C) genes, which have a negative influence on hair growth, decreased by 68%, 80%, and 90% in comparison with the untreated control group. The mRNA expression levels of SRD5A1 (FIG. 3D) and SRD5A2 (FIG. 3E) genes, known as type 1 and type 2 5alpha-reductase, decreased by 60% and 50%.

### DISCLOSURE OF INVENTION

The present disclosure relates to a composition including a mango extract and more preferably a solvent extract of mango leaves as an active ingredient for alleviation, suppression, prevention, or treatment of hair loss, and a preparation method therefor.

1. A method of alleviating, preventing or treating hair loss comprising administering an effective amount of a mango (Mangifera indica) extract as an active ingredient to a subject in need thereof.

2. The method of claim 1, wherein the mango extract is obtained by extraction with one or more solvents selected from the group consisting of water, a straight-chain or branched alcohol having 1 to 4 carbon atoms, acetone, ethyl acetate, butyl acetate, 1,3-butylene glycol, hexane, and diethyl ether.

3. The method of claim 1, wherein the mango extract is a mango leaf extract.

4. The method of claim 3, wherein the mango leaf is an apple mango leaf.

5. A food composition comprising a mango extract as an active ingredient for alleviation or suppression of hair loss.

6. The food composition of claim 5, wherein the mango extract is obtained by extraction with one or more solvents selected from the group consisting of water, a straight-chain or branched alcohol having 1 to 4 carbon atoms, acetone, ethyl acetate, butyl acetate, 1,3-butylene glycol, hexane, and diethyl ether.

7. A cosmetic composition comprising a mango extract as an active ingredient for alleviation or prevention of hair loss.

8. The cosmetic composition of claim 7, wherein the mango extract is obtained by extraction with one or more solvents selected from the group consisting of water, a straight-chain or branched alcohol having 1 to 4 carbon atoms, acetone, ethyl acetate, butyl acetate, 1,3-butylene glycol, hexane, and diethyl ether.
